(19) United States
(12) Patent Application Publication
(10) Pub. No.: US 2004/0097890 A1
(43) Pub. Date: May 20, 2004

(54) Applicator for scalp medicine

(76) Inventor: Bary Wilkinson, San Francisco, CA (US)

Correspondence Address: Bruce H. Johnsonbaugh, Eckhoff & Hoppe, 333 Sacramento Street, San Francisco, CA, 94111, US

(21) Appl. No.: 10/712,698

(22) Filed: Nov. 12, 2003

Related U.S. Application Data
(60) Provisional application No. 60/426,498, filed on Nov. 13, 2002.

(51) Int. Cl. A61M 35/00

(52) U.S. Cl. 604/289

(57) ABSTRACT

An applicator for liquid scalp medicine is provided. The applicator includes a reservoir to which a plurality of hollow tines is connected, each tine having a resilient, flexible tip at its end. An aperture is formed in each of the flexible tips, for adjusting the amount of medicine flowing through the aperture in response to pressure applied by the flexible tip against the scalp. The aperture is a slit formed in the surface of the tip. The aperture closes when no pressure is applied by the tip against the scalp. The flow of medicine is thereby stopped. The applicator may be refillable, and may be either a single unit dose applicator or an applicator capable of carrying multiple doses.

### CROSS-REFERENCE TO RELATED APPLICATION

This application claims the benefit of and priority from U.S. provisional application Serial No. 60/426,498 filed on Nov. 13, 2002.

### BACKGROUND AND BRIEF SUMMARY OF INVENTION

The present invention relates in general to medicinal applicators. More particularly, the present invention relates to an applicator for scalp medicine, for example, Rogaine. The present invention provides a novel, adjustable aperture at the applicator tip whereby small amounts of medicine can be applied directly to the scalp. This is particularly important in applying expensive medicines in order to maximize the effectiveness and efficiency of use.

It is known in the prior art to provide medicated combs with reservoirs wherein the medicine simply flows from the reservoir through openings in a plurality of teeth. These patents include Donley et al U.S. Pat. No. 4,090,522; Lorenzo U.S. Pat. No. 6,035,806; Foreman U.S. Pat. No. 5,555,899 and Yamamoto et al Publication No. US 2001/0040173 A1. Those prior art devices simply allow the medicine to flow from the reservoir in the head of the device through channels in the teeth or tines and onto the scalp. No provision is made, in these prior devices, for intermittently stopping the flow of medicine or for limiting the amount of medicine that flows from the applicator onto the scalp.

There is a need for an applicator that allows the user to apply small quantities of medicine, regulate the amount of medicine that flows from the reservoir through the tines, and to allow the user to interrupt or stop the flow of medicine as desired. This need becomes more compelling as the cost of various scalp medicines, including Rogaine, steadily increases.

There is also a need for an applicator capable of administering a single dose, and, in particular, an applicator which is refillable and small enough to be carried easily in a pocket or small bag.

With the present invention, an applicator for scalp medicines is provided wherein each tine has a novel, adjustable aperture formed at or near its tip. When the applicator is not in use, or when no pressure is applied to the tine, the aperture closes and no medicine flows through it. However, when the user contacts the scalp with the tip of the tines and applies slight pressure, the aperture opens and allows medicine to flow through the tine onto the scalp. The more pressure the user applies, the more the aperture opens, allowing a larger amount of medicine to flow onto the scalp. The present design furthermore reduces substantially the amount of medicine that adheres to the hair, rather than being applied to the scalp. This is accomplished by positioning the aperture directly adjacent the scalp. The invention includes a single or unit dose applicator which in one embodiment is refillable and in another embodiment is disposable.

A primary object of the invention is to provide an applicator for scalp medicines wherein the user is able to apply small amounts of medicine, to limit and even intermittently stop the flow of medicine through the applicator and onto the scalp.

A further object of the invention is to provide an applicator for scalp medicines which achieves optimum application of expensive medicines directly to the scalp, thereby minimizing the amount of medicine that is applied to the hair and minimizing the amount of unintended, excess medicine that is allowed to flow out of the applicator.

A further object of the invention is to provide an applicator having a discharge aperture that automatically closes when no pressure is applied to the applicator by the user, but opens in proportion to the amount of pressure applied to the applicator by the user.

Another object of the invention is to provide a medicinal applicator for the scalp having an adjustable aperture through which the medicine flows, that is easily and intuitively controlled by the user, and requires no moving parts.

Another object of the invention is to provide a single or unit dose applicator, which may be either refillable or disposable.

A further object of the invention is to provide a small, unit dose applicator that is easily carried by the user.

Other objects and advantages of the invention will become apparent from the following detailed description and drawings wherein:

### BRIEF DESCRIPTION OF THE DRAWINGS

FIG. 1 is a front elevational view of one embodiment of the applicator according to the present invention;

FIG. 2 is a side elevational view of the applicator shown in FIG. 1;

FIG. 3 is a bottom view along line 3-3 of FIG. 1;

FIG. 4 is the same bottom view as shown in FIG. 3 when a slight amount of pressure has been applied by the user;

FIG. 5 is the same bottom view shown in FIGS. 3 and 4 when a greater amount of pressure has been applied by the user;

FIG. 6 is a front elevational view of one of the tines illustrated in FIG. 1 showing an alternate form of the invention;

FIG. 7 is a front view of the tine illustrated in FIG. 6 when pressure is applied to the tine;

FIG. 8 is a front view of an alternate form of the invention;

FIG. 9 illustrates the tine of FIG. 8 when pressure is applied by the user;

FIG. 10 is a side elevational view of an alternate form of the invention;

FIG. 11 is a bottom view of the tine shown in FIG. 10, without any pressure applied by the user;

FIG. 12 is the same bottom view as FIG. 11 when pressure is applied by the user;

FIG. 13 is a front, sectional view of an alternate embodiment of the invention;

FIG. 14 is a side elevational view of the applicator of FIG. 13 showing a cover in place over the hollow tines;

FIG. 15 is a front sectional view of a further embodiment of the invention;

FIG. 16 is a sectional view along line 16-16 of FIG. 15;

FIGS. 17 and 18 are schematic representations of one form of a one-way valve used in conjunction with the applicator shown in FIGS. 15 and 16; and

FIGS. 19 and 20 are schematic representations illustrating how the applicator with a one-way valve shown in FIGS. 15 and 16 cooperates with a storage container to refill the applicator.

### DETAILED DESCRIPTION OF THE DRAWINGS

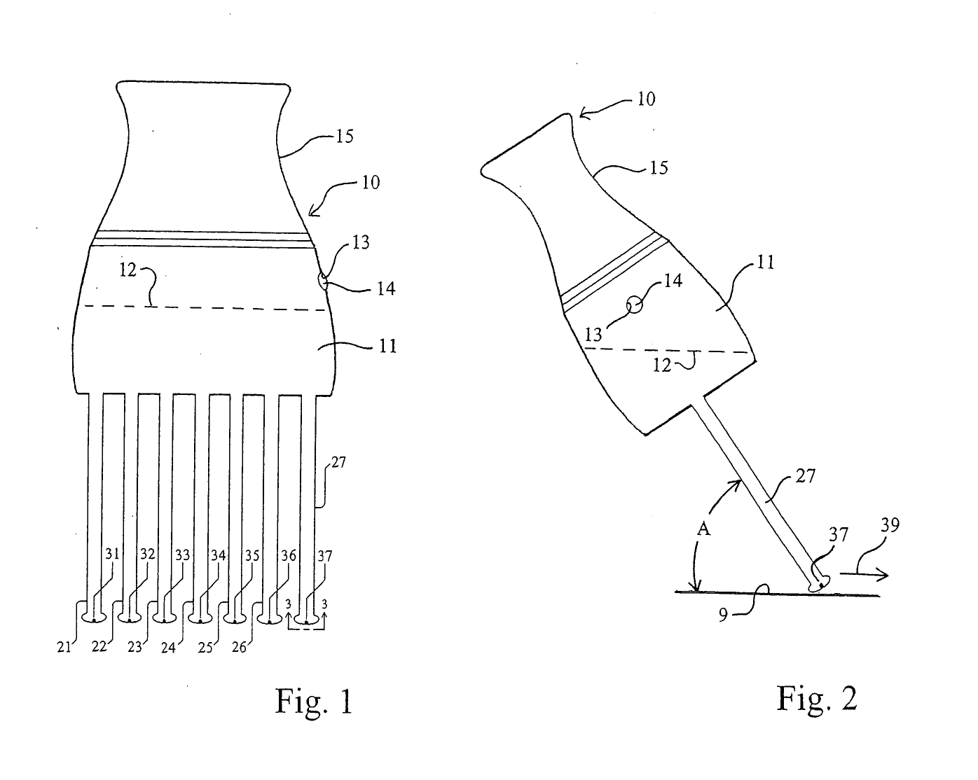

FIGS. 1 and 2 illustrate the applicator shown generally as 10 according to the present invention. A body 11 is formed which acts as a reservoir for holding liquid medicine shown generally as 12. Medicine 12 is inserted into the body 11 through an opening 13 covered with a removable plug 14. The upper portion of the applicator includes a somewhat reduced neck 15 to form a shape easily handled by one hand of a user.

A plurality of tines 21-27 extend downwardly from body 11 and are in fluid communication with liquid medicine 12. The applicator as illustrated in FIG. 2 is in its position of use, wherein tines 21-27 form an angle A with respect to the user's scalp 9 and the tines 21-27 in this embodiment are moved in a first, or forward, direction shown by arrow 39. Tines 21-27 have closed tips, except for adjustable apertures, as described below.

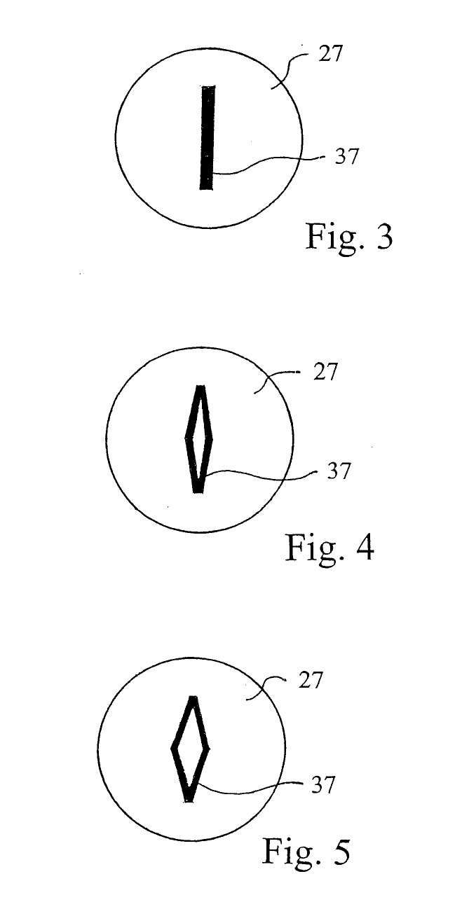

FIG. 3 illustrates a bottom view of tine 27 and illustrates aperture 37. The aperture 37 is formed in the bottom of tine 27 as by laser cutting. Aperture 37 extends through the bottom wall of tine 27. Aperture 37 is illustrated in FIG. 3 in its closed position.

FIG. 4 illustrates aperture 37 when a slight bending pressure has been applied to tine 27 as tine 27 is oriented at an angle A relative to the scalp as shown in FIG. 2, and moved in the direction of arrow 39, as illustrated in FIG. 2. A slight amount of pressure applied by the user to tine 27 against the scalp causes aperture 37 to open slightly, as illustrated in FIG. 4, and as more pressure is applied aperture 37 will open further to the position illustrated in FIG. 5. As the user releases pressure, and releases any bending forces applied to tine 27, the aperture 37 again closes to the position shown in FIG. 3. The use of an adjustable and closable aperture 37 allows the user to apply very small amounts of expensive scalp medicine and to interrupt the flow of medicine as desired. The application of larger bending forces applied to tine 27 increases the amount of medicine flowing through aperture 37. Since aperture 37 is formed at or near the very lower end or tip of tine 27, the medicine is applied directly to the scalp and little or no medicine is applied to the hair and wasted. As the user releases pressure and raises the applicator off the scalp, aperture 37 closes and prevents any more medicine from passing through it until the tine 27 recontacts the user's scalp. Aperture 37 is a slit formed in the tip of tine 27 and extends completely through one wall of the tip.

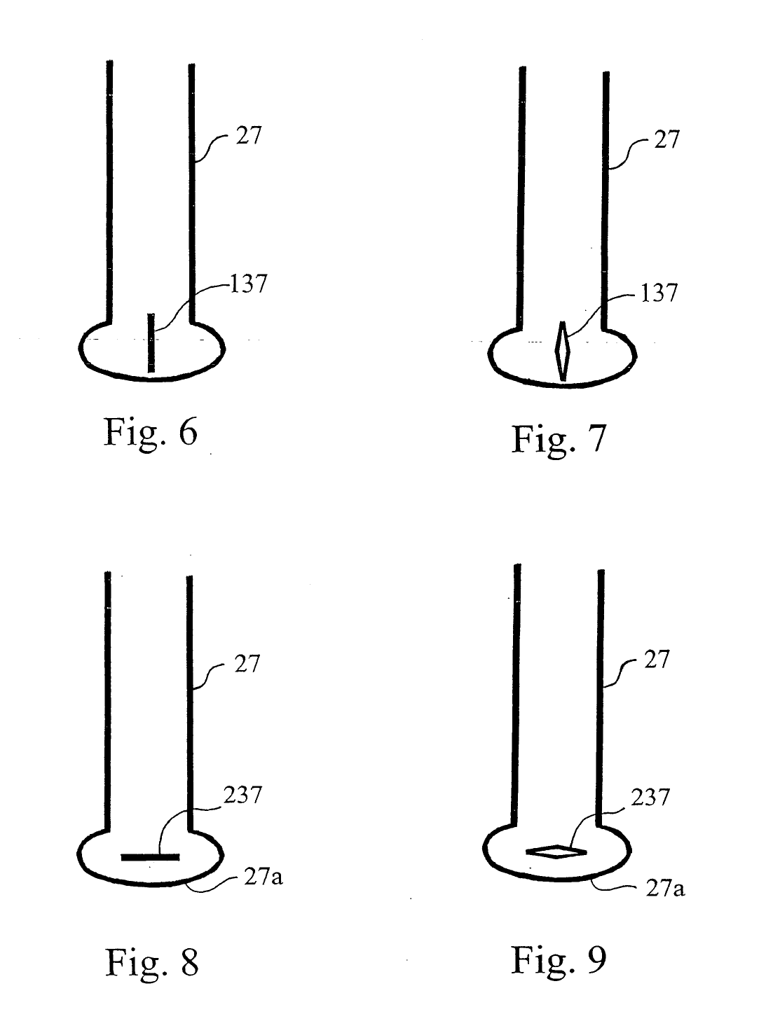

FIGS. 6 and 7 illustrate a second embodiment of the invention as applied to tine 27 illustrated in FIGS. 1 and 2. Rather than having an aperture formed at the very bottom of tine 27, as illustrated in FIGS. 3, 4 and 5, a slitted aperture 137 is formed in only one side of tine 27 near the end of the tine. Aperture 137 extends parallel with the longitudinal axis of tine 27. In this embodiment, as tine 27 is pulled across the scalp with aperture 137 adjacent the scalp, aperture 137 will expand as illustrated in FIG. 7 when slight bending pressure is applied to tine 27.

FIGS. 8 and 9 illustrate another variation of the invention wherein aperture 237 is formed in a direction transverse or perpendicular to the longitudinal axis of tine 27. The transverse aperture 237 is formed at or near the extreme lower tip 27a of tine 27. Again, as the user draws the lower tip 27a across his scalp with aperture 237 on or adjacent to the scalp, slight bending forces applied to tine 27 will cause aperture 237 to open in response to the amount of bending force.

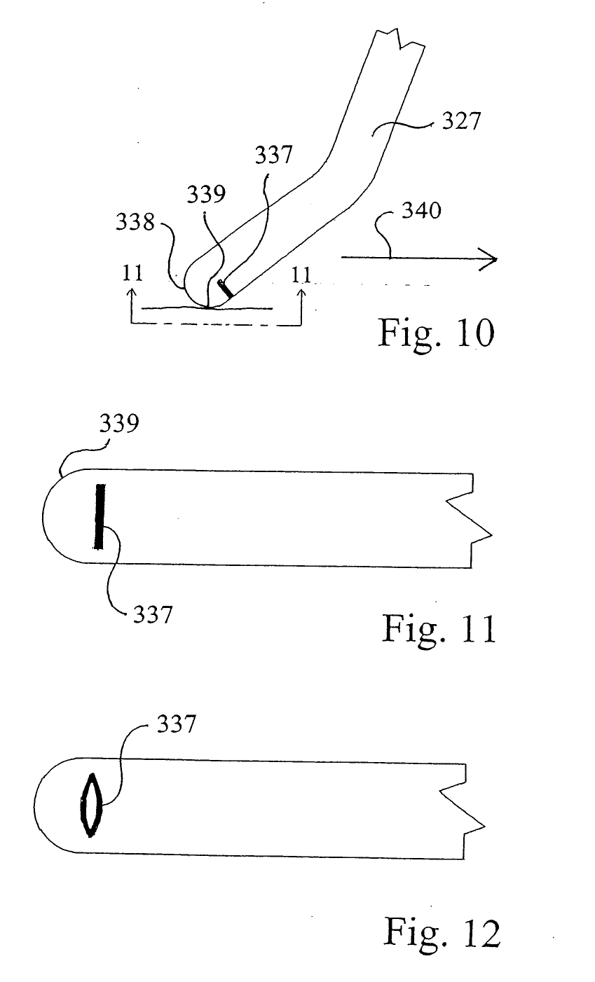

FIGS. 10-12 illustrate yet an additional embodiment of the invention. In this embodiment, the tine 327 has a lowermost tip 338. Tine 327 has a circular cross section and is curved along its longitudinal length. In operation, tine 327 is held relative to the scalp so that point 339, near the extreme lowermost tip 338, physically contacts the scalp. A transverse aperture 337 is formed adjacent point of contact 339 and between point 339 and the body of the applicator. As illustrated in FIG. 12, as tine 327 is pulled in the direction of arrow 340 (FIG. 10), aperture 337 opens to the position shown in FIG. 12, releasing medicine. Aperture 337 is positioned at or near the tip of the tine, preferably between the point of contact with the scalp and the body of the applicator.

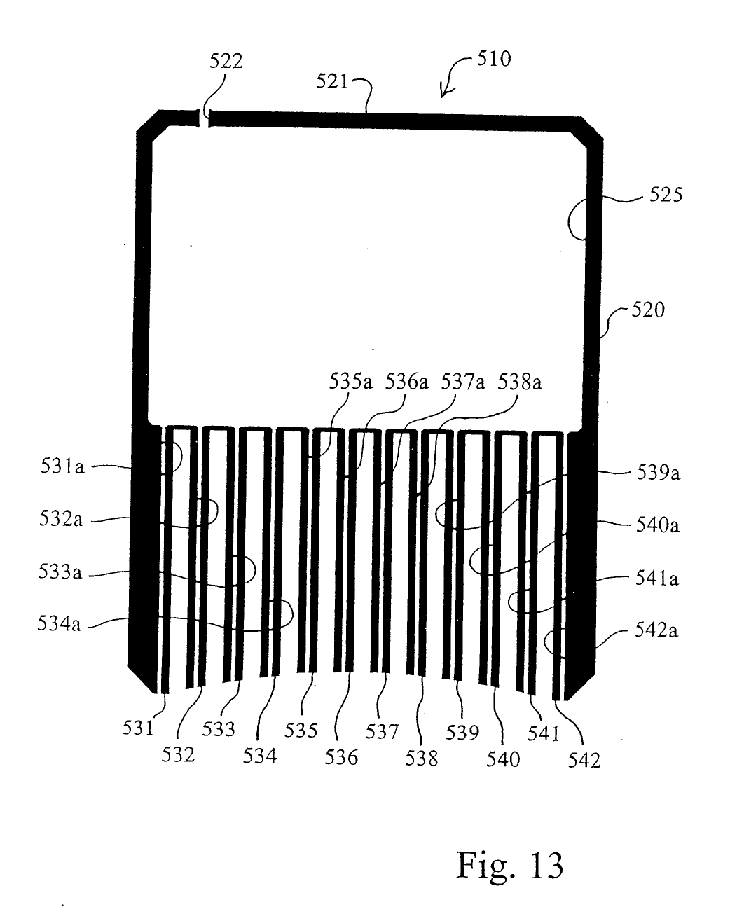

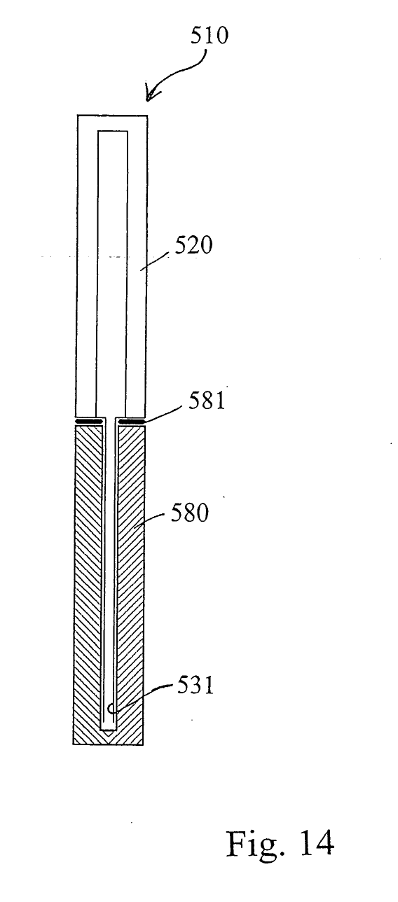

FIGS. 13 and 14 illustrate an alternate embodiment of the invention wherein a disposable, single dose applicator is provided for scalp medicines. FIG. 13 shows the applicator 510 with cover 580 (FIG. 14) removed. Applicator 510 includes a hollow body 520 with a generally rectangular shape. Hollow body 520 contains a reservoir 525 for carrying scalp medicine. The top 521 of the body 520 has an opening 522 formed therein that allows the reservoir 525 to be filled with scalp medicine. A plug (not shown) is pressed into the opening 522 after the reservoir 525 has been filled. A plurality of hollow tines 531-542 is carried by the body 520. Each of the tines 531-542 contains a capillary action sized passageway 531a-542a. The tips of the tines 531-542 are open, forming passageways 531a-542a that extend through the tips of the tines. Passageways 531a-542a are in fluid communication with the reservoir 525, have uniform cross-sections, and are sized to facilitate capillary flow of the liquid scalp medicine through the passageways directly onto the scalp.

FIG. 14 is a side elevational view of the applicator 510 shown in FIG. 13, but with cover 580 in place over tines 531-542. A seal 581 is provided between the cover 580 and the body 520.

To use the device shown in FIGS. 13 and 14, the user simply removes the cover 580 and presses the tines 531-542 lightly against the scalp. The passageways 531a-542a are sized to provide capillary flow of liquid scalp medicine when the tips of the tines 531-542 contact the user's scalp. The reservoir 525 is sized to carry one dose, a single dose requiring approximately one milliliter of medicine in the case of Rogaine. Reservoir 525 may be made larger or smaller for single doses of other medicine. The applicator 510 may be used once and discarded. It is small enough and thin enough to be carried in a pocket, purse or in larger wallets.

The tines are made of resilient, flexible material, such as plastic. The applicator body and tines are preferably injection molded. Other materials may be utilized as well.

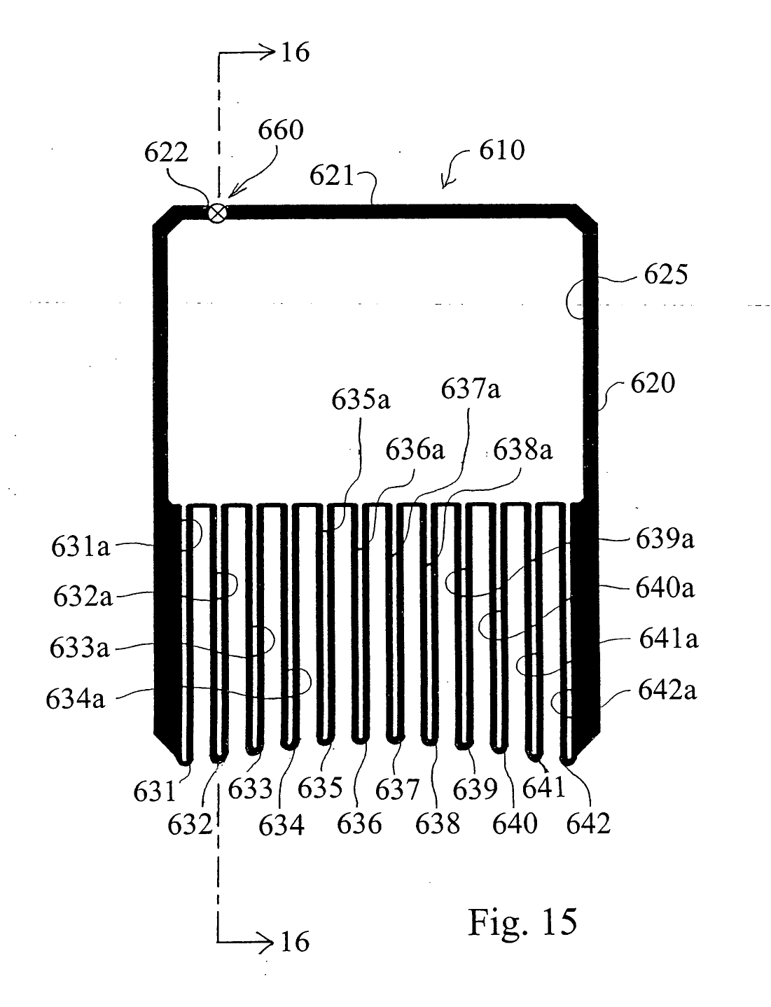

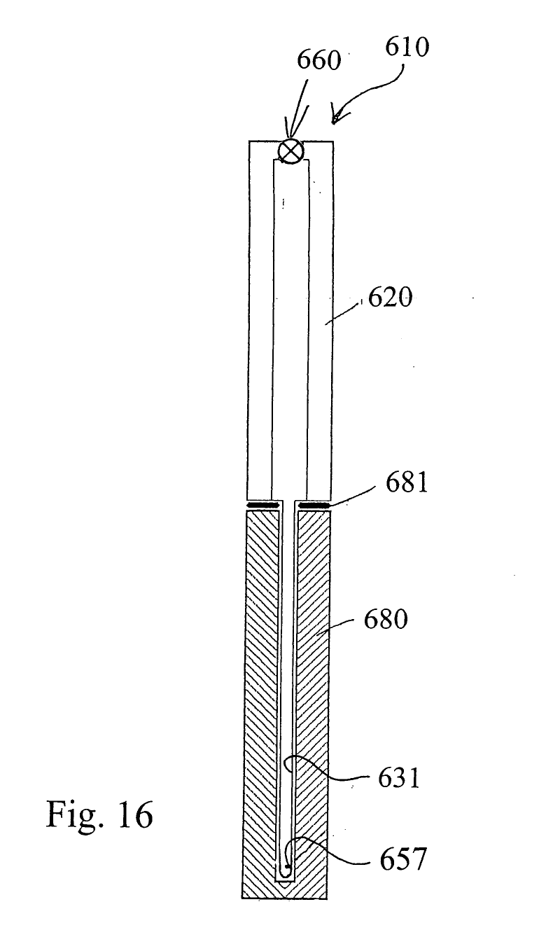

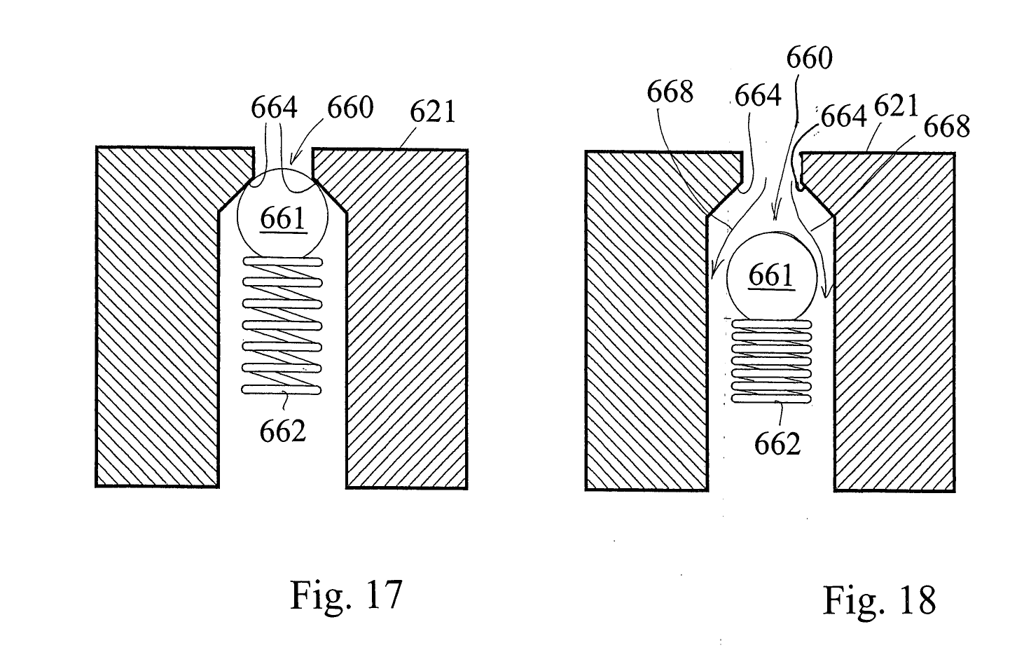

FIGS. 15 and 16 illustrate a further embodiment of the invention. The applicator 610 is refillable. In addition, it may be sized to hold multiple doses or sized to hold only a single or unit dose. The tines 631-642 are closed at the tips except for apertures formed in the tips as shown in FIGS. 10-12 and described above. Aperture 657 is shown in FIG. 16. The hollow body 620 has a reservoir 625 formed in its interior for carrying scalp medicine. The top 621 of the body 620 has an opening 622 which carries a one-way valve means 660. The one-way valve means 660 may include a variety of valves known in the prior art. For example, one-way valve means 660 may comprise a spring loaded ball valve 660 as illustrated in FIGS. 17 and 18. A spherical ball 661 is carried on a resilient spring 662. The ball 661 in its first or closed position, as shown in FIG. 17, is held against a valve seat 664 formed in the top 621 of the body 620. When fluid pressure is applied to the top of the ball 661, the spring 662 compresses and allows fluid to flow past the one-way valve 660 as shown by arrows 668. FIG. 18 illustrates the second or open position of the ball 661 in which Rogaine, for example, is allowed to flow past the ball 661 into the reservoir 625.

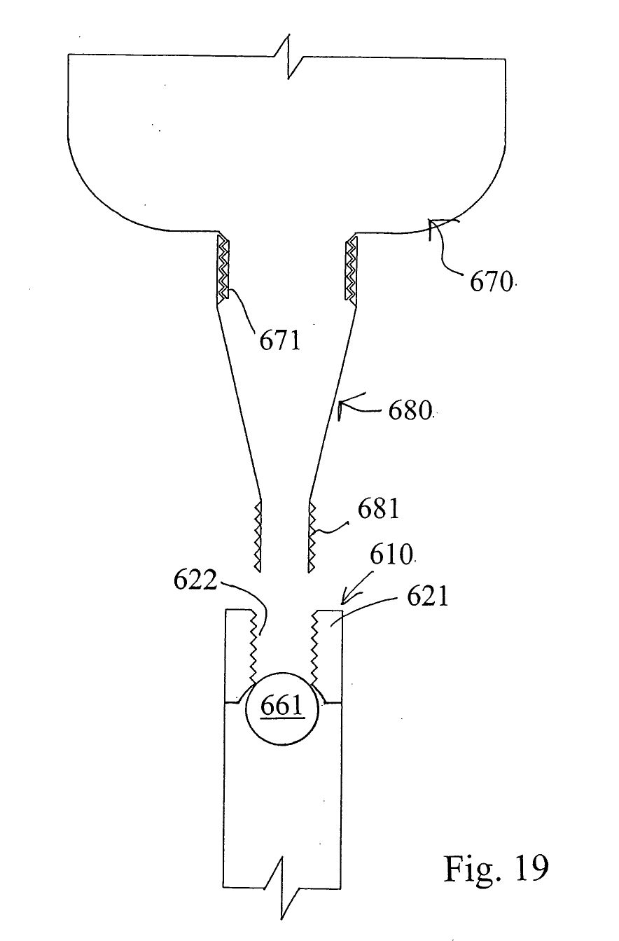

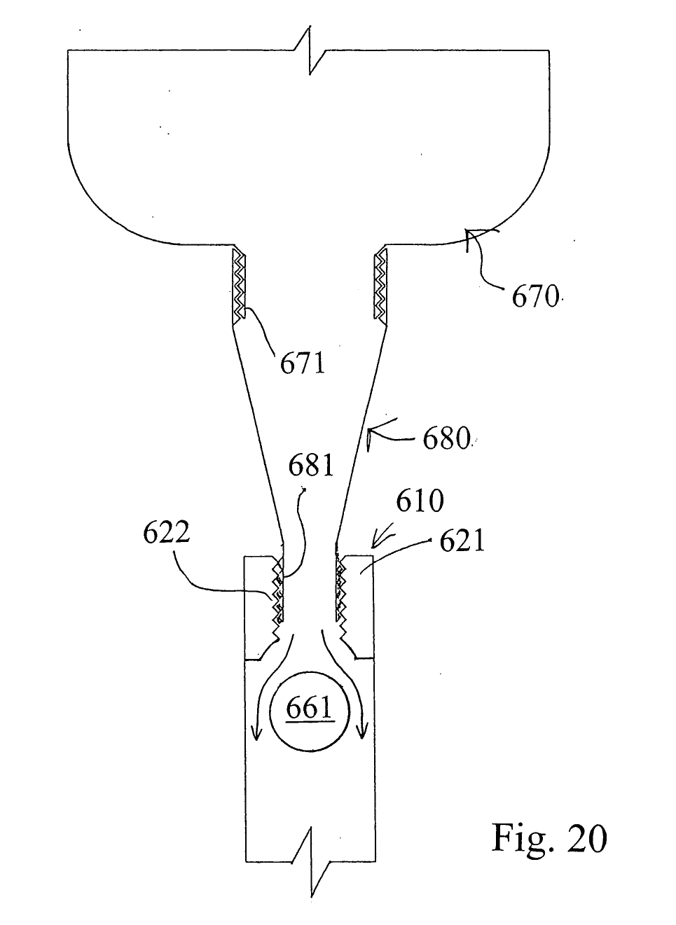

FIGS. 19 and 20 are schematic representations showing how the embodiment shown in FIG. 15 may be refilled. A rather large container 670 is shown which carries, for example, a three month or six month supply of Rogaine or other scalp medicine. The container 670 is shown in FIG. 19 in an inverted position. The container 670 has a threaded opening 671 which is normally covered with a flat cap (not shown in FIG. 19, for brevity). When the user desires to fill the applicator 610, he simply removes the ordinary storage cap from the container 670, applies filler cap 680 and fills the container 610 as described below. The container 670 is held upright (the reverse of the position shown in FIG. 19) and the distal tip 681 of the filler cap is threaded into the opening 622 of the applicator 610. The storage container 670 is then inverted and squeezed. The fluid pressure opens the ball valve 661 as described above and the applicator 610 is filled. The adjustable apertures function as vents during the filling process, allowing air to escape as liquid flows into the tines. The supply container 670 is then inverted to an upright position and the applicator 610 is unthreaded from the tip 681. An optional threaded plug may be inserted threaded into the opening 622 of the applicator 610 to assure no leakage. The tapered filler cap 680 is then removed from the storage container 670 and an ordinary cap is threaded onto the thread 671. Optionally, a threaded cap may be threaded onto the tip 681 to seal the medicine in the container 670.

The invention can also be used to dispense other medications, such as lice medication, dandruff medication and medications for other scalp conditions.

The foregoing description of the invention has been presented for purposes of illustration and description and is not intended to be exhaustive or to limit the invention to the precise form disclosed. Modifications and variations of the above are possible in light of the above teaching. These particular embodiments were chosen and described to best explain the principles of the invention and its practical application, thereby enabling others skilled in the art to best use the invention in various embodiments and with various modifications suited to the particular use contemplated. The scope of the invention is to be defined by the following claims.

What is claimed is:

1. An applicator for liquid scalp medicine, comprising:
a reservoir for holding a supply of said liquid scalp medicine,
a plurality of hollow tines carried by said reservoir, said hollow tines having passageways in fluid contact with said reservoir,
a resilient, flexible and closed tip at the end of each of said hollow tines, and
adjustable aperture means formed in or near each of said tips for adjusting the amount of said liquid scalp medicine that flows through said aperture means in response to pressure applied by said tip against the scalp, wherein said aperture means includes a slit formed in said tip extending completely through one wall of said tip and wherein said adjustable aperture means closes when no pressure is applied by said tip against the scalp, thereby stopping the flow of said liquid scalp medicine.

2. The apparatus of claim 1 wherein each of said tines has a longitudinal axis and wherein said slit is parallel to said longitudinal axis.

3. The apparatus of claim 1 wherein each of said tines has a longitudinal axis and wherein said slit is transverse to said longitudinal axis.

4. The apparatus of claim 1 further comprising one-way valve means connected to said reservoir whereby said applicator is refillable.

5. The apparatus of claim 4 wherein said reservoir is sized to carry a single dose of said liquid scalp medicine.

6. An applicator for liquid scalp medicine, comprising:
a reservoir for holding a supply of said liquid scalp medicine,
a plurality of hollow tines carried by said reservoir, said hollow tines having passageways in fluid contact with said reservoir,
a resilient, flexible and closed tip at the end of each of said hollow tines, and
each of said hollow tines has a passageway of uniform cross-section, said passageway extending through said open tip of each tine, said passageway being sized to facilitate capillary flow through it by said liquid scalp medicine.
